(19) United States
(12) Patent Application Publication
(10) Pub. No.: US 2019/0176131 A1
(43) Pub. Date: Jun. 13, 2019

(54) Methods of Making Supported Mixed Metal Dehydrogenation Catalysts

(71) Applicant: ExxonMobil Chemical Patents Inc., Baytown, TX (US)

(72) Inventors: Doron Levin, Highland Park, NJ (US); William A. Lamberti, Stewartsville, NJ (US)

(21) Appl. No.: 16/174,372

(22) Filed: Oct. 30, 2018

Related U.S. Application Data
(60) Provisional application No. 62/597,190, filed on Dec. 11, 2017.

(51) Int. Cl. B01J 23/62; B01J 37/08; B01J 37/06; B01J 37/02; B01J 27/135; B01J 21/12; C07C 5/367; C07C 5/10; C07C 2/74; C07C 5/32

(52) U.S. Cl. CPC B01J 23/626; B01J 37/08; B01J 37/06; B01J 37/0205; B01J 37/0236; B01J 27/135; C07C 2523/62; C07C 5/367; C07C 5/10; C07C 2/74; C07C 5/325; C07C 2521/08; B01J 21/12

(57) ABSTRACT

Disclosed herein is are methods of preparing dehydrogenation catalysts comprising the steps of calcining a catalyst precursor in an oxygen-containing atmosphere followed by a calcining the calcined catalyst precursor in a hydrogen-containing atmosphere and/or washing the calcined catalyst precursor with water. The dehydrogenation catalysts prepared in accordance with the methods of the present disclosure typically comprise a halogen content of less than 0.1 wt % based on the weight of the dehydrogenation catalyst. Such catalysts may be particularly useful in the dehydrogenation of a feed comprising cyclohexane and/or methylcyclopentane.

### CROSS-REFERENCE TO RELATED APPLICATION

This application claims priority from U.S. Provisional Application No. 62/597,190, filed Dec. 11, 2017, which is incorporated herein by reference.

### FIELD

The present invention relates to dehydrogenation catalysts, methods of making the same, and processes using the same for dehydrogenating saturated cyclic hydrocarbons such it) as cyclohexane and/or methylcyclopentane.

### BACKGROUND

Phenol and cyclohexanone are important compounds in the chemical industry and are useful in, for example, production of phenolic resins, bisphenol A, ε-caprolactam, adipic acid, and plasticizers.

A common route for the production of phenol is the Hock process. This is a three-step process in which the first step involves alkylation of benzene with propylene to produce cumene, followed by oxidation of cumene to the corresponding hydroperoxide, and then cleavage of the hydroperoxide to produce equimolar amounts of phenol and acetone. The separated phenol product can then be converted to cyclohexanone by a step of hydrogenation.

Cyclohexylbenzene can be produced by contacting benzene with hydrogen in the presence of a bifunctional catalyst comprising a molecular sieve of the MCM-22 type and at least one hydrogenation metal selected from palladium, ruthenium, nickel, cobalt, and mixtures thereof. This reference also discloses that the resultant cyclohexylbenzene can be oxidized to the corresponding hydroperoxide, which can then be cleaved to produce a cleavage mixture of phenol and cyclohexanone, which, in turn, can be separated to obtain pure, substantially equimolar phenol and cyclohexanone products.

One disadvantage of this cyclohexylbenzene-based process is that it produces impurities such as cyclohexane and methylcyclopentane. These impurities represent loss of valuable benzene feed. Moreover, unless removed, these impurities will tend to build up in the system, thereby displacing benzene and increasing the production of undesirable by-products. Thus, a significant problem facing the commercial application of cyclohexylbenzene as a phenol precursor is removing the cyclohexane and methylcyclopentane impurities.

The use of a metal halide as the metal source can result in the presence of halogens in various forms, e.g., halides, particularly chlorides, on the finished catalyst, e.g., resulting in a chlorine concentration of greater than 0.10 wt % by weight of the finished catalyst. Although the halogen content of the finished catalyst can be reduced during activation, the resulting release of halogen species, e.g., chlorides and HCl formed during activation, into the reactor system is undesirable for a variety of reasons. For example, in such aspects the metallurgy of the reactor and downstream equipment needs to be suitable to avoid halogen, e.g., chloride/HCl, induced corrosion, thereby limiting the ability to use carbon steel. Additionally, it is typically desirable to remove released halogen species, e.g., HCl, from the system, either via purging, which results in a loss of production, or via adsorption with additional equipment, which increases the cost of the dehydrogenation. Accordingly, it would be advantageous to provide a dehydrogenation catalyst having a low halogen content prior to or in lieu of activation.

Moreover, methods of regulating the halogen content in catalyst compositions comprising metal deposited on an alumina support are typically not suitable for catalyst compositions comprising metal, e.g., platinum, deposited on a silica-containing support due to agglomeration of the metal.

Thus, in order to solve the above-mentioned problems, there is a need for dehydrogenation catalyst compositions that are substantially free of halogen, e.g., chlorine, and other impurities, as well as methods of making the same and dehydrogenation processes using the same.

References of interest include U.S. Pat. Nos. 9,580,368, 9,469,580, 7,579,511, 6,037,513, 3,852,217, WO 2009/131769, and, WO 2011/096998.

### SUMMARY

The present disclosure relates to a method of preparing a dehydrogenation catalyst, the method comprising (or consisting of, or consisting essentially of) the steps of: a) providing a catalyst precursor comprising (i) an inorganic support comprising silica (ii) a first metal selected from Group 14 of the Period Table of Elements and (iii) a second metal selected from Groups 6 to 10 of the Periodic Table of Elements; and b) calcining the catalyst precursor at a temperature of from 200° C. to 700° C. in an oxygen-containing atmosphere to obtain a first calcined catalyst precursor, wherein the method further comprises one of the following steps c) through f): c) calcining the calcined catalyst precursor at a temperature ranging from 150° C. to 600° C. in a hydrogen-containing atmosphere to obtain the dehydrogenation catalyst; d) washing the calcined catalyst precursor with water at a temperature of below 100° C. to obtain the dehydrogenation catalyst; e) calcining the calcined catalyst precursor at a temperature ranging from 150° C. to 600° C. in a hydrogen-containing atmosphere, followed by washing the calcined catalyst precursor with water at a temperature of below 100° C. to obtain the dehydrogenation catalyst; or f) washing the calcined catalyst precursor with water at a temperature of below 100° C., followed by calcining the calcined catalyst precursor at a temperature ranging from 150° C. to 600° C. in a hydrogen-containing atmosphere to obtain the dehydrogenation catalyst.

The present disclosure also relates to a dehydrogenation catalyst comprising (or consisting of, or consisting essentially of) (i) from 0.05 to 5 wt % of a first metal oxide based on the weight of the dehydrogenation catalyst, wherein the first metal oxide comprises a metal selected from Group 14 of the Periodic Table of Elements and (ii) from 0.1 to 10 wt % of a second metal oxide selected based on the weight of the dehydrogenation catalyst, wherein the second metal oxide comprises a metal selected from Groups 6-10 of the Period Table of Elements, wherein the first metal oxide and the second metal oxide are deposited on a silica-containing support, and wherein the dehydrogenation catalyst has a halogen content of less than 0.1 wt % based on the weight of the dehydrogenation catalyst.

The present disclosure also relates to a dehydrogenation process employing a dehydrogenation catalyst of the present disclosure. A hydrogenation feed suitable for use in the dehydrogenation process of the present disclosure can be obtained by a) contacting benzene and hydrogen with a hydroalkylation catalyst under hydroalkylation conditions effective to convert benzene to cyclohexylbenzene and cyclohexane; and b) separating at least a portion of the cyclohexane from step a) to form the dehydrogenation feed.

Additional features and advantages of the invention will be set forth in the detailed description and claims, as well as the appended drawings. It is to be understood that the foregoing general description and the following detailed description are merely exemplary of the invention, and are intended to provide an overview or framework to understanding the nature and character of the invention as it is claimed.

### DETAILED DESCRIPTION

As discussed above, the present disclosure is directed towards methods for preparing a dehydrogenation catalyst. The methods of the present disclosure have at least one or more of the following advantages. First, by utilizing the methods described herein, the resulting dehydrogenation catalyst is substantially free of halogen, i.e., comprises a halogen content of less than 0.1 wt % based on the weight of the dehydrogenation catalyst. Accordingly, typical problems arising from activation of a conventional dehydrogenation catalyst, e.g., corrosion of process equipment and/or added costs of processing steps to remove halogen, may be reduced or eliminated through the methods described herein. Second, by utilizing the methods described herein, other impurities apart from halogen, particularly ionic species such as sodium, calcium, sulfur, lithium, magnesium, etc., can also be significantly removed from the catalyst composition, which further increases the resulting performance of the dehydrogenation catalyst.

In the present disclosure, a process is described as comprising at least one “step.” It should be understood that each step is an action or operation that may be carried out once or multiple times in the process, in a continuous or discontinuous fashion. Unless specified to the contrary or the context clearly indicates otherwise, each step in a process may be conducted sequentially in the order as they are listed, with or without overlapping with one or more other step, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are performed in the order as listed.

When a process is said to “consist essentially of” steps or other features, this means that there are no other major steps that will influence the final product such as addition of a reactant that would become part of the final product, but there may be other minor steps such as solvent removal/addition, heating/cooling, mixing, catalyst addition that does not become part of the final product, and other steps that may either not change the resulting product or enhance its yield.

Unless otherwise indicated, all numbers indicating quantities in the present disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contain a certain level of error due to the limitation of the technique and equipment used for making the measurement.

As used herein, the indefinite article “a” or “an” shall mean “at least one” unless specified to the contrary or the context clearly indicates otherwise. Thus, embodiments using “a hydrogenation metal” include embodiments where one, two, or more different types of the hydrogenation metals are used, unless specified to the contrary or the context clearly indicates that only one type of the hydrogenation metal is used.

As used herein, “wt %” means percentage by weight, “vol %” means percentage by volume, “mol %” means percentage by mole, “ppm” means parts per million, and “ppm wt” and “wppm” are used interchangeably to mean parts per million on a weight basis. All “ppm” as used herein are ppm by weight unless specified otherwise. All concentrations herein are expressed on the basis of the total amount of the composition in question. All ranges expressed herein should include both end points as two specific embodiments unless specified or indicated to the contrary.

Unless otherwise specified, as used herein, a composition that is “substantially free” of a component means that the component is present at a concentration of less than 0.1 wt % by weight of the composition.

As used herein, the generic term “dicylcohexylbenzene” includes, in the aggregate, 1,2-dicyclohexylbenzene, 1,3-dicylohexylbenzene, and 1,4-dicyclohexylbenzene, unless clearly specified to mean only one or two thereof. The term cyclohexylbenzene, when used in the singular form, means mono substituted cyclohexylbenzene.

In the present disclosure, the composition of catalysts and precursors of catalysts are expressed on the basis of the dry components. To the extent that the catalyst materials may entrain water, such water is not considered in its composition. While the catalyst materials or their precursors may be processed and/or used with a small quantity of water contained therein, it is preferred that the catalyst is dry (e.g., having a water content of at most 5.0 wt %, or at most 3.0 wt %, or at most 1.0 wt %, or at most 0.5 wt %, or at most 0.1 wt %) when put into use in a dehydrogenation process according to the present disclosure. Unless otherwise specified, in the present disclosure, the quantities of the first, second and third metals in the catalysts and precursors thereof are expressed on the basis of elemental metal, regardless of the oxidation state thereof. Thus, all quantities of Pt, Pd, Sn, K, Na, Ni, Co, and other Groups 1, 2, 6-10, and 14 metals in these catalyst materials are expressed on an elemental basis, even though they may be present in the materials at issue in the form of, e.g., in whole or in part, salts, oxides, complexes, and elemental metals. For example, a catalyst composition made with 1.9 grams of tin chloride salt (1.0 gram of tin) and 22.29 grams of tetraamine platinum hydroxide solution (4.486 wt % Pt) that is supported on 98 grams of silicon dioxide contains 1.0 wt % of tin and 1.0 wt % Pt, based upon the weight of the catalyst composition in dry components. Also, the composition of the precursor of a catalyst is expressed in terms of the final composition of the catalyst prepared therefrom. One having ordinary skill in the art of catalyst preparation can batch the starting materials, such as salts, solutions, oxides, and the like, to achieve a final target chemical composition of the catalyst. For example, in the present disclosure, a catalyst precursor comprising 1.0 wt % of Pt, 1.0 wt % Sn, and 98.0 wt % of silica means a precursor comprising the desired amount of starting materials such as one or more of PtO₂, Pt, SnO₂, SnO, SnCl₄, SnCl₂, and the like, that upon activation and/or other treatments described herein, would be converted into a final catalyst comprising platinum, tin, and silica in the above amounts.

Unless otherwise indicated, the amount of a given component present in a catalyst composition and/or catalyst precursor described herein, e.g., metal, halogen, sodium, sulfur, or other ionic species content, is determined via Wavelength Dispersive X-Ray Fluorescence spectroscopy (“XRF”) using a Bruker S8 Tiger Wavelength Dispersive X-ray Fluorescence (WDXRF) 3 kW System Spectrometer, wherein the sample is ground into a powder prior to analysis.

As used herein, the numbering scheme for the Periodic Table Element Groups disclosed herein is the New Notation provided on the inside cover of Hawley's Condensed Chemical Dictionary (14th Edition), by Richard J. Lewis.

The term “MCM-22 type material” (or “material of the MCM-22 type” or “molecular sieve of the MCM-22 type” or “MCM-22 type zeolite”), as used herein, includes one or more of:
molecular sieves made from a common first degree crystalline building block unit cell, which unit cell has the MWW framework topology. A unit cell is a spatial arrangement of atoms which if tiled in three-dimensional space describes the crystal structure. Such crystal structures are discussed in the “Atlas of Zeolite Framework Types,” Fifth Edition, 2001, the entire content of which is incorporated as reference;
molecular sieves made from a common second degree building block, being a 2-dimensional tiling of such MWW framework topology unit cells, forming a monolayer of one unit cell thickness, desirably one c-unit cell thickness;
molecular sieves made from common second degree building blocks, being layers of one, or more than one, unit cell thickness, wherein the layer of more than one unit cell thickness is made from stacking, packing, or binding at least two monolayers of one unit cell thickness. The stacking of such second degree building blocks can be in a regular fashion, an irregular fashion, a random fashion, or any combination thereof; and
molecular sieves made by any regular or random 2-dimensional or 3-dimensional combination of unit cells having the MWW framework topology.

Molecular sieves of the MCM-22 type include those molecular sieves having an X-ray diffraction pattern including d-spacing maxima at 12.4±0.25, 6.9±0.15, 3.57±0.07, and 3.42±0.07 Angstrom. The X-ray diffraction data used to characterize the material are obtained by standard techniques such as using the K-alpha doublet of copper as incident radiation and a diffractometer equipped with a scintillation counter and associated computer as the collection system.

Materials of the MCM-22 type include MCM-22 (described in U.S. Pat. No. 4,954,325), PSH-3 (described in U.S. Pat. No. 4,439,409), SSZ-25 (described in U.S. Pat. No. 4,826,667), ERB-1 (described in European Patent No. 0293032), ITQ-1 (described in U.S. Pat. No. 6,077,498), ITQ-2 (described in WO 97/17290), MCM-36 (described in U.S. Pat. No. 5,250,277), MCM-49 (described in U.S. Pat. No. 5,236,575), and MCM-56 (described in U.S. Pat. No. 5,362,697). Other molecular sieves, such as UZM-8 (described in U.S. Pat. No. 6,756,030), may be used alone or together with the MCM-22 type molecular sieves as well for the purpose of the present disclosure. Desirably, the molecular sieve is selected from (a) MCM-49; (b) MCM-56; and (c) isotypes of MCM-49 and MCM-56, such as ITQ-2.

### Catalyst Preparation Methods

The present disclosure provides methods of preparing dehydrogenation catalysts that are substantially free of halogen species, e.g., chlorine, by utilizing the steps of calcining a catalyst precursor in an oxygen-containing atmosphere, followed by additional high temperature calcination precursor in a hydrogen-containing atmosphere and/or washing the calcined catalyst precursor with water, preferably deionized water. Advantageously, the methods of the present disclosure are further effective in decreasing the content of other undesired ionic species in the prepared catalyst composition, e.g., sodium and sulfur.

Typically, the catalyst precursor comprises (i) an inorganic support; (ii) a first metal selected from Group 14 of the Periodic Table of Elements, preferably tin; (iii) a second metal selected from Groups 6 to 10 of the Periodic Table of Elements, preferably platinum and/or palladium; and optionally (iv) a third metal selected from Groups 1 and 2 of the Periodic Table of Elements. For example, the catalyst precursor may comprise both the first metal and the second metal, but is essentially free of the third metal.

The inorganic support may comprise one or more of silica, alumina, a silicate, an aluminosilicate, zirconia, carbon, or carbon nanotubes. Alternatively, the support may comprise an inorganic oxide such as one or more of silicon dioxide, titanium dioxide, and zirconium dioxide. Preferably, the support is a silica-containing support and comprises less than 0.5 wt % of alumina. More preferably, the support is substantially free of or free of alumina. The support may or may not comprise a binder. Impurities that can be present in the catalyst support are, for example, sodium salts such as sodium silicate, which can be present from anywhere from 0.01 wt % to 2 wt % based on the weight of the support. Suitable silica supports are described in, for example, WO2007/084440 A1.

Suitable silica-containing supports typically have pore volumes and median pore diameters determined by the method of mercury intrusion porosimetry described by ASTM D4284. The silica support may have surface areas as measured by ASTM D3663. The pore volumes may be in the range from 0.2 cc/gram to 3.0 cc/gram. The median pore diameters are in the range from 10 angstroms to 2000 angstroms, or from 20 angstroms to 500 angstroms; and the surface areas (m²/gram) are in the range from 10 to 1000 m²/gram, or from 20 to 500 m²/gram.

The first metal, second metal, and optional third metal may be deposited on the inorganic support to form the catalyst precursor via any method known in the art, preferably by impregnating the support with a solution containing the metal.

Suitable solutions containing the first, second, or optional third metal can be prepared by dissolving a source of the first second, or third metal, or a precursor thereof, in a solution carrier, e.g., water. Optionally, an organic dispersant may be added to assist in uniform application of the metal component(s) to the support. Suitable organic dispersants include organic acids, such as citric acid and amino alcohols and amino acids, such as arginine. For example, the organic dispersant may be present in the solution composition in an amount between 1.0 wt % and 20 wt % of the solution composition.

Useful metal sources or precursors are not particularly limited, and can comprise oxides, halides, carbonates, sulfides, hydrides, or hydroxides of the first metal, second metal, or optional third metal. For example, stannous chloride can be used as a tin source, and tetraamine platinum hydroxide can be used as a platinum source.

The impregnation of the first, second, and optional third metals can be conducted at a temperature of less than 100° C., for example, at ambient temperature (i.e., from 20 to 25° C.) for a time period of at least 0.1, 0.2, 0.5, 0.8, 1.0, 1.5, 2.0, 3.0, 4.0, 5.0, 6.0, 7.0, 8.0, 10.0, 12.0, 15.0, 20.0, 24.0, 30.0, 36.0, or 48.0 hours, preferably from 0.5 hours to 2 hours.

Typically, following each impregnation step of the first, second, and optional third metal, the impregnated catalyst support is subjected to a drying step. The drying steps may be carried out at a temperature of less than 200° C., for example, from 80 to 200° C., or from 100 to 180° C., or from 120 to 150° C. to remove the water or other solution carrier, and organic dispersant, if present. Preferably, the dried impregnated inorganic support may contain less than 5.0, 4.5, 4.0, 3.0, 2.0, 1.5, or 1.0 wt % of water by weight of the impregnated dried support. The drying steps may be accomplished by any technique known to those skilled in the art effective for removal of water or other solution carrier, for example exposure to heated air, vacuum drying at a pressure below atmospheric pressure, or microwave drying.

In a particularly preferred embodiment, the catalyst precursor can be prepared by the following steps: impregnating a silica-containing support with a first solution containing a first metal selected from Group 14 of the Periodic Table of Elements to obtain a first impregnated support; drying the first impregnated support at a temperature of below 200° C. to obtain a first dried support; impregnating the first dried support with a second solution containing at least one second metal selected from Groups 6 to 10 of the Periodic Table of Elements to obtain a second impregnated support; and drying the second impregnated support at a temperature of below 200° C. to obtain the catalyst precursor.

Generally, the catalyst precursor prepared in accordance with any of the above described methods is subjected to a first calcination step in an oxygen-containing atmosphere. Preferably, this calcination step is conducted at a temperature ranging from 200 to 700° C. for a time of 0.5 to 50 hours, such as at least 0.5, 0.8, 1, 3, 5, 10, 16, 20, 24, 30, 36, 40, or 48 hours. Typically, the calcination is effective with respect to one or more of the following: (i) removal of the solution carrier; (ii) conversion of metal salt(s) to metal oxide(s); and (iii) decomposing the organic dispersant. The first calcination step is typically conducted in an oxidizing atmosphere, such as air.

The first calcined catalyst precursor is generally subjected to a second calcination step in a hydrogen-containing atmosphere, a washing step, or a combination of the foregoing in order to obtain the dehydrogenation catalyst.

The second calcination step can be conducted at a temperature of from 150 to 600° C., for example, from 200 to 600° C., or from 250 to 525° C., or from 350 to 500° C., or from 400 to 500° C. The second calcination step can be carried out for a time period of at least 0.1, 0.2, 0.3, 0.5, 0.8, 1, 3, 5, 10, 16, 20, 24, 30, 36, 40, or 48 hours up to 100, 90, 80, 72, or 60 hours. Often, the second calcination step is carried out for a time period of at least 3 hours, preferably at least 10 hours, or at least 15 hours. Typically, the second calcination step is further effective with respect to one or more of the following: (i) removal of the solution carrier; (ii) conversion of metal oxide(s) to an activated form; and (iii) decomposing the organic dispersant.

The washing step can be conducted with water, preferably deionized water, at a temperature of less than 100° C., for example, from 20 to 95° C., or from 30 to 80° C. Without wishing to be bound by theory, it is believed that the effectiveness of the washing step in removing impurities improves with increasing temperature. Preferably, the washing step can be conducted more than one time, e.g., twice. Preferably each washing steps is performed fresh water, preferably deionized water.

Generally, the dehydrogenation catalyst prepared in accordance with the above methods, e.g., subjected to a second calcination and/or at least one washing step, has a reduced content of halogens and/or other impurities as compared to the first calcined catalyst precursor. Preferably, the dehydrogenation catalyst may have a halogen content, e.g., chlorine content, of less than 0.10, 0.095, 0.090, 0.085, 0.08, 0.075, 0.07, 0.065, 0.06, 0.055, 0.05, 0.045, 0.04, 0.035, or 0.03 wt % based on the weight of the dehydrogenation catalyst. Additionally or alternatively, the dehydrogenation catalyst may contain:

(i) less than 0.15, 0.13, 0.12, 0.118, 0.115, 0.1, 0.095, 0.09, 0.085, 0.08, 0.075, 0.07, 0.065, 0.06, 0.05, 0.04, 0.03, or 0.025 wt % of sulfur based on the weight of the dehydrogenation catalyst; and/or

(ii) less than 0.5, 0.45, 0.4, 0.35, 0.3, 0.25, 0.2, 0.15, or 0.1 wt % of sodium based on the weight of the dehydrogenation catalyst.

Particularly advantageously, the dehydrogenation catalyst may be free or substantially free of halogens, sulfur, and/or sodium. Additionally, the herein described preparation methods can be readily used to remove other impurities, such as K, F, Ca, Mg, Li, etc. that may typically be present in metal supported catalyst compositions.

Preferably, the second calcination and/or washing step(s) described above selectively reduce the impurity content, e.g., halogen content, of the catalyst without reducing the metal content. For example, preferably the first and/or second metal content in the prepared dehydrogenation catalyst is greater than 80%, preferably greater than 90%, more preferably greater than 95%, and ideally greater than 99% of the first and/or second metal content in the first calcined catalyst precursor.

In any embodiment, in the dehydrogenation catalyst prepared in accordance with the methods of the present disclosure, the metal selected from Group 14 is typically present in an amount in the range of between any two of the following percentages: 0.01 wt %, 0.03 wt %, 0.05 wt %, 0.08 wt %, 0.1 wt %, 0.15 wt %, 0.3 wt %, 0.5 wt %, 0.8 wt %, 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, and 10 wt %, for example from 0.01 wt % to 10 wt %, or from 0.05 wt % to 5.00 wt % based on the weight of the dehydrogenation catalyst.

In any embodiment, in the dehydrogenation catalyst prepared in accordance with the methods of the present disclosure, the metal selected from Groups 6 to 10 is typically present in an amount in a range between any two of the following percentages: 0.01 wt %, 0.02 wt %, 0.03 wt %, 0.05 wt %, 0.08 wt %, 0.1 wt %, 0.3 wt %, 0.5 wt %, 0.8 wt %, 1 wt %, 1.5 wt %, 2 wt %, 2.5 wt %, 3 wt %, 3.5 wt %, 4 wt %, 4.5 wt %, or 5 wt %, 5.5 wt %, 6 wt %, 6.5 wt %, 7 wt %, 7.5 wt %, 8 wt %, 8.5 wt %, 9 wt %, 9.5 wt %, and 10 wt %, for example from 0.01 wt % to 10 wt %, or from 0.1 wt % to 5 wt % based on the weight of the dehydrogenation catalyst.

Alternatively or additionally, the dehydrogenation catalyst may comprise (i) nickel at a concentration of at most 2.0 wt %, or at most 1.0 wt %, or at most 0.5 wt %, or at most 0.1 wt % nickel; and (ii) cobalt at a concentration of at most 2.0 wt %, or at most 1.0 wt %, or at most 0.5 wt %, or at most 0.1 wt %, the percentages based on the weight of the dehydrogenation catalyst. Preferably, the catalyst composition is free or substantially free of ruthenium, rhodium, lead, and/or germanium, and/or any other active elemental components.

The ratio of the second metal selected from Groups 6 to 10 of the Periodic Table of Elements to the first metal selected from Group 14 of the Periodic Table of Elements (e.g., the Pt/Sn ratio) in the catalyst can be in a range between any two of the following values: 0.1, 0.2, 0.3, 0.4, 0.5, 0.8, 1.0, 2.0, 2.5, 3.0, 4.0, 5.0, 8.0, 10.0, 15.0, 20.0, 30.0, 40.0, 50.0, 60.0, 70.0, 80.0, 90.0, 100, 150, 200, 250, 300, 350, and 400, for example from 2.5 to 400; from 2.5 to 200; and from 3.0 to 100.

It will be understood that the dehydrogenation catalyst prepared in accordance with the methods of the present disclosure may comprise the metal(s) in one or more oxidation states, such as in elemental form, a salt, an oxide, and the like. Typically, following the calcination of the catalyst precursor in an oxygen-containing atmosphere, at least a part of the metal(s) are in an oxidized form. For example, if the catalyst comprises Pt and Sn, the calcined catalyst precursor would typically comprise at least a part of Pt in the form of PtO₂, and a part of Sn in the form of SnO₂. Typically, where the first calcined catalyst precursor is subjected to a second calcination step in a hydrogen-containing atmosphere, said metal oxide(s) are reduced to the metal(s) in elemental form. For example, if the catalyst comprises Pt and Sn, the second calcined catalyst precursor would typically comprise at least a part of Pt in the form of elemental Pt, and a part of Sn in the form of elemental Sn.

It will further be understood that washing the catalyst precursor with water generally has no effect on the oxidation state of the metal(s). Thus, where the dehydrogenation catalyst is prepared by subjecting the first calcined catalyst precursor to one or more washing steps in the absence of the second calcination step, the dehydrogenation catalyst typically comprises a Group 14 metal oxide and a Group 6-10 metal oxide. In such aspects, the dehydrogenation catalyst generally comprises (i) from 0.05 to 5 wt % of a first metal oxide based on the weight of the dehydrogenation catalyst, wherein the first metal oxide comprises a metal selected from Group 14 of the Periodic Table of Elements and (ii) from 0.1 to 10 wt % of a second metal oxide based on the weight of the dehydrogenation catalyst, wherein the second metal oxide comprises a metal selected from Groups 6-10 of the Periodic Table of Elements.

The dehydrogenation catalyst may be subject to a step of activation prior to being used in a dehydrogenation reaction. Alternatively, the activation step may be omitted, particularly if the metal(s) are in elemental form (e.g., where the first calcined catalyst precursor is subjected to a second calcination step in a hydrogen-containing atmosphere). The activation step typically involves heating the catalyst in a reducing atmosphere comprising H₂ at an elevated temperature. The reducing atmosphere can be pure hydrogen, or a mixture of hydrogen with other reducing or inert gas, such as N₂, CH₄, C₂H₅, other hydrocarbons, and the like. Preferably, the H₂-containing atmosphere used for the activation step prior to contacting the catalyst is a substantially dry stream of gas comprising H₂O at no more than 5.0, 4.0, 3.0, 2.0, 1.0, 0.8, 0.5, 0.3, 0.1, 0.05, 0.01, 0.005, 0.001, 0.0005, or even 0.0001 vol %. The dry H₂ stream can serve to heat the catalyst precursor, dry the precursor before significant reduction occurs, and purging the H₂O produced during reduction, if any. Upon contacting with hydrogen at high temperature, the first metal, if in an oxidation state higher than elemental, would be at least partly reduced to a lower oxidation state, advantageously elemental state. For example, PtO₂ and PdO can be reduced to Pt and Pd by H₂ at an elevated temperature. The second metal, if in an oxidation state higher than elemental, may be reduced to a lower oxidation state or elemental state as well in the activation step by hydrogen and/or other components in the activation atmosphere. The third metal, however, being a Group 1 or 2 metal in the Periodic Table such as K, Na, Ca, and the like, if present in the catalyst, would most likely remain in an oxidation state higher than elemental in the activated catalyst in the form of oxide, salt, or part of complex material such as a glass or ceramic material formed with the inorganic support material.

During the activation step, the catalyst may be heated from a lower temperature, e.g., room temperature (23° C.), to a target activation temperature. As used herein, “activation temperature” means the highest temperature the catalyst is exposed to for at least 3 minutes (or at least 5 minutes, or at least 10 minutes, or at least 15 minutes, or at least 20 minutes) during activation. It is highly desired that the catalyst is surrounded by a H₂-containing atmosphere during the heating step. When the temperature is relatively low, e.g., lower than 100° C., reducing of the first and/or second and/or third metal(s) can be slow and negligible. The higher the temperature of the catalyst precursor, the higher the rate of the reducing reactions. Thus, it is desired that the highest temperature the catalyst is exposed to (and thus, reached) during the activation step is not lower than 300, 320, 340, 350, 360, 380, 400, 420, 440, or 450° C. It is highly desired that the catalyst is held within the temperature range (“activation temperature”) from T_{act}−20° C. to T_{act} for an activation duration of at least D1 minutes, where T_{act} is the activation temperature, and D1 can be 10, 15, 20, 25, 30, 45, 60, 75, 90, 120, 150, 180, 240, 300, 360, 420, 480, 540, 600, 660, 720, 780, 840, or even 900. It has been found that too high an activation temperature and too long a temperature hold period around the maximal temperature can be detrimental to the performance of the activated catalyst. Without intending to be bound by a particular theory, it is believed that the first and second metals in elemental form may be mobilized on the surface of the inorganic support at very high temperature, agglomerate to form large crystals, thereby reducing the number of effective sites on the activated catalyst. Thus, it is desired that the activation temperature the catalyst is exposed to in the activation step is not higher than 650, 640, 630, 620, 610, 600, 590, 580, 570, 560, or 550° C. It is desired that during the heating step and the temperature hold period, the catalyst is exposed to a H₂-containing atmosphere at least b % of the time, where “b” can be 50, 60, 70, 80, 90, 95, 98, or even 100%. When and if the catalyst is not surrounded by an H₂-containing atmosphere, it is highly desired that it is surrounded by an otherwise reducing or inert atmosphere, such as CH₄, N₂, and mixtures thereof, and the like.

At the end of the temperature holding period around the activation temperature, it is desired that at least 40%, 50%, 55%, 60%, 65%, 70%, 75%, 80%, 85%, 90%, 95%, 98%, 99%, 99.5%, 99.8%, or even 99.9% of all of the first and second metals in the catalyst have been reduced to the desired oxidation state, such as elemental state. Where the catalyst comprises Pt and/or Pd, it is desired that at least 50%, 55%, 60%, 65%, 70%, 75%, 80%, 85%, 90%, 95%, 98%, 99.5% or even 99.8% of Pt and/or Pd are reduced to elemental Pt and Pd at the end of the temperature holding period. Where the catalyst comprises Sn, it is desired that at least 40%, 45%, 50%, 55%, 60%, 65%, 70%, 75%, 80%, 85%, 90%, 95%, 98%, or even 99% of Sn is reduced to elemental Sn at the end of the temperature holding period.

The activated dehydrogenation catalyst may have an oxygen chemisorption value (ocv) of greater than 5, 8, 12, 15, 18, 20, 22, 25, 28, or 30. As used herein, the oxygen chemisorption value (ocv) of a particular catalyst is a measure of metal dispersion on the catalyst and is defined as:

$$O C V = \frac{amount of oxygen sorbed by the catalyst in moles}{\begin{matrix}amount of dehydrogenation metal \\ contained in the catalyst in moles\end{matrix}} \times 100 \% .$$

The oxygen chemisorption values referred to herein are measured using the Micromeritics ASAP 2010 physisorption analyzer as follows. Approximately 0.3 to 0.5 grams of catalyst are placed in the Micrometrics device. Under flowing helium, the catalyst is ramped from ambient (18° C.) to 250° C. at a rate of 10° C. per minute and held for 5 minutes. After 5 minutes, the sample is placed under vacuum at 250° C. for 30 minutes. After 30 minutes of vacuum, the sample is cooled to 35° C. at 20° C. per minute and held for 5 minutes. The oxygen and hydrogen isotherm is collected in increments at 35° C. between 0.50 and 760 mm Hg. Extrapolation of the linear portion of this curve to zero pressure gives the total (i.e., combined) adsorption uptake.

Preferably, the alpha value of the dehydrogenation catalyst is from 0 to 10, such as from 0 to 5, or from 0 to 1. The alpha value of the support is an approximate indication of the catalytic cracking activity of the catalyst compared to a standard catalyst. The alpha test gives the relative rate constant (rate of normal hexane conversion per volume of catalyst per unit time) of the test catalyst relative to the standard catalyst which is taken as an alpha of 1 (Rate Constant=0.016 s⁻¹). The “alpha test” is described in U.S. Pat. No. 3,354,078 and in 4 J. CATALYSIS 527 (1965); 6 J. CATALYSIS 278 (1966); and 61 J. CATALYSIS 395 (1980), to which reference is made for a description of the test. The experimental conditions of the test used to determine the alpha values referred to in this specification include a constant temperature of 538° C., and a variable flow rate as described in detail in 61 J. CATALYSIS (1980). Alternatively, the alpha value may range from vₐᵢₚₕₐ₁ to vₐₗₚₕₐ₂, where vₐₗₚₕₐ₁ can be 0.0, 0.1, 0.2, 0.3, 0.4, 0.5, 0.6, 0.7, 0.8, 0.9, 1, 2, 3, 4, 5, 6, 7, 8, 9, and 10; and vₐₗₚₕₐ₂ can be 200, 175, 150, 125, 100, 90, 80, 70, 60, 50, 40, 30, 20, 10, 5, 1.9, 1.8, 1.7, 1.6, 1.5, 1.4, 1.3, 1.2, 1.1, 1, 0.9, 0.8, 0.7, 0.6, and 0.5, as long as vₐₗₚₕₐ₁<vₐₗₚₕₐ₂.

### Dehydrogenation Process

The activated dehydrogenation catalyst made by using the method of the present disclosure can be used for dehydrogenating a first composition comprising any dehydrogenable hydrocarbon materials such as those containing a cyclic hydrocarbon compound. Preferably, the first composition comprises a cyclic hydrocarbon compound, such as cyclopropane, cyclobutane, cyclopentane, cyclohexane, cycloheptane, cyclo octane, cyclododecane, cyclodecane, cycloundecane, and derivatives (such as alkylated derivatives) thereof. The first composition may comprise C1 wt % to C2 wt % of a saturated cyclic hydrocarbon (e.g., cyclohexane), where C1 and C2 can be, independently, 0.1, 0.2, 0.3, 0.4, 0.5, 0.6, 0.7, 0.8, 0.9, 1, 3, 5, 8, 1, 15, 2, 25, 30, 35, 40, 45, 50, 55, 60, 70, 75, 80, 85, 90, 95, 98, as long as C1<C2, where the percentages are based on the weight of the first composition contacting the activated catalyst.

Where the first composition comprises a six-membered cyclic hydrocarbon such as cyclohexane, it may further comprise one or more five-membered ring cyclic hydrocarbon (such as cyclopentane, methylcyclopentane, ethylcyclopentane, and the like), at a concentration based on the total weight of the first composition in a range from C3 wt % to C4 wt %, where C3 and C4 can be, independently, 0.01, 0.03, 0.05, 0.08, 0.10, 0.15, 0.20, 0.25, 0.30, 0.35, 0.40, 0.45, 0.5, 0.6, 0.7, 0.8, 0.0, 1, 1.5, 2, 2.5, 3, 3.5, 4, 4.5, 5, as long as C3<C4.

The first composition may further comprise a non-dehydrogenable component, such as an aromatic hydrocarbon, at a concentration based on the total weight of the first composition in a range from C5 wt % to C6 wt %, where C5 and C6 can be, independently, 5, 8, 10, 15, 20, 25, 30, 35, 40, 45, 50, 55, 60, 65, 70, 75, 80, 85, 90, or 95, as long as C5<C6. The aromatic hydrocarbon may be, for example, benzene. The aromatic hydrocarbon can be the same as the product of the dehydrogenation process using the activated dehydrogenation catalyst of the present disclosure. The non-dehydrogenable component in the first composition can serve as the heat carrier needed for maintaining the dehydrogenation reaction at a desired temperature and reaction rate.

Suitable conditions for the dehydrogenation step include a temperature of 100° C. to 1000° C., a pressure of atmospheric to 100 kPa-gauge to 7000 kPa-gauge (kPag), and a weight hourly space velocity of 0.2 hr⁻¹ to 50 hr⁻¹.

Preferably, the temperature of the dehydrogenation process can be from T_{d1}° C. to T_{d2}° C., where T_{d1} can be 100, 150, 200, 250, 300, 350, 400, 450, 500, 550, 600; and T_{d2} can be 1000, 950, 900, 850, 800, 750, 700, 650, 600, or 550, as long as T_{d1}<T_{d2}.

Preferably, the pressure of the dehydrogenation process can be from P1 kPa (gauge) to P2 kPa (gauge), where P1 and P2 can be, independently, 0, 100, 150, 200, 250, 300, 350, 400, 450, 500, 600, 700, 800, 900, 1000, 1500, 2000, 2500, 3000, 4000, 5000, 6000, or 7000 so long as the P1 is not greater than P2.

The reactor configuration used for the dehydrogenation process may comprise one or more fixed bed reactors containing a solid catalyst with a dehydrogenation function. Per-pass conversion of the saturated cyclic hydrocarbon (e.g., cyclohexane) using the present catalyst can be at 40, 50, 60, 70, 75, 80, 85, 90, 95, or even 98% conversion. The reaction is endothermic. Typically temperature of the reaction mixture drops across a catalyst bed because of the endothermic effect. External heat may be supplied through one or more heat exchangers to the reactant in the reactor to maintain the temperature of the reactant in the desired range. The temperature of the reaction composition drops across each catalyst bed, and then is raised by the heat exchangers. Preferably, 1 to 5 beds are used, with a temperature drop of 30° C. to 100° C. across each bed. Preferably, the last bed in the series runs at a higher exit temperature than the first bed in the series.

Although the present process can be used with any composition comprising a saturated cyclic hydrocarbon (e.g., cyclohexane) and, optionally a five-membered ring compound (e.g., methylcyclopentane), the process has particular application as part of an integrated process for the conversion of benzene to phenol. In such an integrated process the benzene is initially converted to cyclohexylbenzene by any conventional technique, including alkylation of benzene with cyclohexene in the presence of an acid catalyst, such as zeolite beta or an MCM-22 type molecular sieve, or by oxidative coupling of benzene to biphenyl followed by hydrogenation of the biphenyl. However, in practice, the cyclohexylbenzene is generally produced by contacting the benzene with hydrogen under hydroalkylation conditions in the presence of a hydroalkylation catalyst whereby the benzene undergoes the following reaction (1) to produce cyclohexylbenzene (CHB):

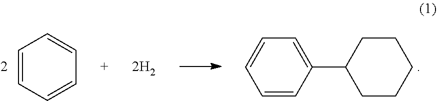 [SMILES: c1ccccc1 ; c1ccc(C2CCCCC2)cc1]

The hydroalkylation reaction can be conducted in a wide range of reactor configurations including fixed bed, slurry reactors, and/or catalytic distillation towers. In addition, the hydroalkylation reaction can be conducted in a single reaction zone or in a plurality of reaction zones, in which at least the hydrogen is introduced to the reaction in stages. Suitable reaction temperatures are from 100° C. to 400° C., such as from 125° C. to 250° C., while suitable reaction pressures (gauge) are from 100 kPa to 7,000 kPa, such as from 500 kPa to 5,000 kPa. Suitable values for the molar ratio of hydrogen to benzene are between 0.15:1 and 15:1, such as between 0.4:1 and 4:1 for example, between 0.4:1 and 0.9:1.

The catalyst employed in the hydroalkylation reaction is generally a bifunctional catalyst comprising a molecular sieve of the MCM-22 type described above and a hydrogenation metal.

Any known hydrogenation metal can be employed in the hydroalkylation catalyst, although suitable metals include palladium, ruthenium, nickel, zinc, tin, and cobalt, with palladium being particularly advantageous. The amount of hydrogenation metal present in the catalyst can be in a range from 0.05 wt % to 10 wt %, such as from 0.1 wt % to 5.0 wt %, of the catalyst. Where the MCM-22 type molecular sieve is an aluminosilicate, the amount of hydrogenation metal present is such that the molar ratio of the aluminum in the molecular sieve to the hydrogenation metal is preferably from 1.5 to 1500, for example, from 75 to 750, such as from 100 to 300.

The hydrogenation metal may be directly supported on the MCM-22 type molecular sieve by, for example, impregnation or ion exchange. Preferably, at least 50 wt %, for example at least 75 wt %, and generally substantially all of the hydrogenation metal is supported on an inorganic oxide separate from, but composited with the molecular sieve. In particular, it is found that by supporting the hydrogenation metal on the inorganic oxide, the activity of the catalyst and its selectivity to desired products such as cyclohexylbenzene and dicyclohexylbenzene are increased as compared with an equivalent catalyst in which the hydrogenation metal is supported on the molecular sieve.

The inorganic oxide employed in such a composite hydroalkylation catalyst is not narrowly defined provided it is stable and inert under the conditions of the hydroalkylation reaction. Suitable inorganic oxides include oxides of Groups 2, 4, 13, and 14 of the Periodic Table of Elements, such as alumina, titania, and/or zirconia.

The hydrogenation metal can be deposited on the inorganic oxide, such as by impregnation, before the metal-containing inorganic oxide is composited with the molecular sieve. Typically, the catalyst composite can be produced by co-pelletization, in which a mixture of the molecular sieve and the metal-containing inorganic oxide are formed into pellets at high pressure (generally 350 kPa to 350,000 kPa), or by co-extrusion, in which a slurry of the molecular sieve and the metal-containing inorganic oxide, optionally together with a separate binder, are forced through a die. If necessary, additional hydrogenation metal can subsequently be deposited on the resultant catalyst composite. Alternatively, the molecular sieve, the inorganic oxide, and the optional binder can be composited and formed into pellets by, e.g., extrusion, which is then impregnated by the one or more dispersions, such as solutions, containing one or more of the metals.

The catalyst may comprise a binder. Suitable binder materials include synthetic or naturally occurring substances as well as inorganic materials such as clay, silica, and/or metal oxides. The latter may be either naturally occurring or in the form of gelatinous precipitates or gels including mixtures of silica and metal oxides. Naturally occurring clays which can be used as a binder include those of the montmorillonite and kaolin families, which families include the subbentonites and the kaolins, commonly known as Dixie, McNamee, Ga., and Florida clays or others in which the main mineral constituent is halloysite, kaolinite, dickite, nacrite, or anauxite. Such clays can be used in the raw state as originally mined or initially subjected to calcination, acid treatment, or chemical modification. Suitable metal oxide binders include silica, alumina, zirconia, titania, silica-alumina, silica-magnesia, silica-zirconia, silica-thoria, silica-beryllia, silica-titania, as well as ternary compositions such as silica-alumina-thoria, silica-alumina-zirconia, silica-alumina-magnesia, and silica-magnesia-zirconia.

Although the hydroalkylation step is highly selective towards cyclohexylbenzene, the effluent from the hydroalkylation reaction will normally contain unreacted benzene feed, some dialkylated products, and other by-products, particularly cyclohexane, and methylcyclopentane. In fact, typical selectivities to cyclohexane and methylcyclopentane in the hydroalkylation reaction are 1 to 25 wt % and 0.1 to 2.0 wt %, respectively.

The dehydrogenation reaction can be performed on all or a portion of the output of the hydroalkylation step.

Alternatively, the hydroalkylation reaction effluent is separated into at least a (i) C6-rich composition; and (ii) the remainder of the hydroalkylation reaction effluent. When a composition is described as being “rich in” in a specified species (e.g., C6-rich, benzene-rich or hydrogen-rich), it is meant that the wt % of the specified species in that composition is enriched relative to the feed composition (i.e., the input). A “C6” species generally means any species containing 6 carbon atoms.

Given the similar boiling points of benzene, cyclohexane, and methylcyclopentane, it is difficult to separate these materials by distillation. Thus, a C6-rich composition comprising benzene, cyclohexane, and methylcyclopentane may be separated by distillation from the hydroalkylation reaction effluent. This C6-rich composition can be then subjected to the dehydrogenation process described above such that at least a portion of the cyclohexane in the composition is converted to benzene and at least a portion of the methylcyclopentane is converted to linear and/or branched paraffins, such as 2-methylpentane, 3-methylpentane, n-hexane, and other hydrocarbon components such as isohexane, C5 aliphatics, and C1 to C4 aliphatics. The dehydrogenation product composition may then be fed to a further separation system, typically a further distillation tower, to divide the dehydrogenation product composition into a benzene-rich stream and a benzene-depleted stream. The benzene-rich stream can then be recycled to the hydroalkylation step, while the benzene-depleted stream can be used as a fuel for the process. When a composition is described as being “depleted” with respect to a particular species (e.g., benzene-depleted), it is meant that the wt % of the specified species in that composition is depleted relative to the feed composition (i.e., the material charged into the reactor).

After separation of the C6-rich composition, the remainder of hydroalkylation reaction effluent may be fed to a second distillation tower to separate the monocyclohexylbenzene product (e.g., cyclohexylbenzene) from any dicyclohexylbenzene and other heavies. Depending on the amount of dicyclohexylbenzene present in the reaction effluent, it may be desirable to transalkylate the dicyclohexylbenzene with additional benzene to maximize the production of the desired monoalkylated species.

Transalkylation with additional benzene may be effected in a transalkylation reactor, separate from the hydroalkylation reactor, over a suitable transalkylation catalyst, including large pore molecular sieves such as a molecular sieve of the MCM-22 type, zeolite beta, MCM-68 (see U.S. Pat. No. 6,014,018), zeolite Y, zeolite USY, and mordenite. A large pore molecular sieve may have an average pore size of at least 7 Å, such as from 7 Å to 12 Å. The transalkylation reaction is typically conducted under at least partial liquid phase conditions, which suitably include a temperature of 100° C. to 300° C., a pressure of 800 kPa to 3500 kPa, a weight hourly space velocity of 1 hr⁻¹ to 10 hr⁻¹ on total feed, and a benzene/dicyclohexylbenzene weight ratio of 1:1 to 5:1. The transalkylation reaction effluent can then be returned to the second distillation tower to recover the additional monocyclohexylbenzene product produced in the transalkylation reaction.

After separation in the second distillation tower, the cyclohexylbenzene can be converted into phenol and cyclohexanone by a process similar to the Hock process. In this process, cyclohexylbenzene is initially oxidized to the corresponding hydroperoxide. This is accomplished by introducing an oxygen-containing gas, such as air, into a liquid phase containing the cyclohexylbenzene. Unlike the Hock process, atmospheric air oxidation of cyclohexylbenzene, in the absence of a catalyst, is very slow and hence the oxidation is normally conducted in the presence of a catalyst.

Suitable catalysts for the cyclohexylbenzene oxidation step are the N-hydroxy substituted cyclic imides described in U.S. Pat. No. 6,720,462 and incorporated herein by reference, such as N-hydroxyphthalimide, 4-amino-N-hydroxyphthalimide, 3-amino-N-hydroxyphthalimide, tetrabromo-N-hydroxyphthalimide, tetrachloro-N-hydroxyphthalimide, N-hydroxyhetimide, N-hydroxyhimimide, N-hydroxytrimellitimide, N-hydroxybenzene-1,2,4-tricarboximide, N,N′-dihydroxy(pyromellitic diimide), N,N′-dihydroxy(benzophenone-3,3′,4,4′-tetracarboxylic diimide), N-hydroxymaleimide, pyridine-2,3-dicarboximide, N-hydroxysuccinimide, N-hydroxy(tartaricimide), N-hydroxy-5-norbornene-2,3-dicarboximide, exo-N-hydroxy-7-oxabicyclo [2.2.1]hept-5-ene-2,3-dicarboximide, N-hydroxy-cis-cyclohexane-1,2-dicarboximide, N-hydroxy-cis-4-cyclohexene-1,2 dicarboximide, N-hydroxynaphthalimide sodium salt, or N-hydroxy-o-benzenedisulphonimide. Preferably, the catalyst is N-hydroxyphthalimide. Another suitable catalyst is N,N′,N″-trihydroxyisocyanuric acid.

These materials can be used either alone or in the presence of a free radical initiator and can be used as liquid-phase, homogeneous catalysts or can be supported on a solid carrier to provide a heterogeneous catalyst. Typically, the N-hydroxy substituted cyclic imide or the N,N′,N″-trihydroxyisocyanuric acid is employed in an amount between 0.0001 wt % to 15 wt %, such as between 0.001 wt % to 5.0 wt %, of the cyclohexylbenzene.

Suitable conditions for the oxidation step include a temperature between 70° C. and 200° C., such as 90° C. to 130° C., and a pressure of 50 kPa to 10,000 kPa. Any oxygen-containing gas, preferably air, can be used as the oxidizing agent. The reaction can take place in batch reactors or continuous flow reactors. A basic buffering agent may be added to react with acidic by-products that may form during the oxidation. In addition, an aqueous phase may be introduced, which can help dissolve basic compounds, such as sodium carbonate.

Another reactive step in the conversion of the cyclohexylbenzene into phenol and cyclohexanone involves cleavage of the cyclohexylbenzene hydroperoxide, which is conveniently effected by contacting the hydroperoxide with a catalyst in the liquid phase at a temperature of 20° C. to 150° C., such as 40° C. to 120° C., and a gauge pressure of 50 kPa to 2,500 kPa, such as 100 kPa to 1000 kPa. The cyclohexylbenzene hydroperoxide is preferably diluted in an organic solvent inert to the cleavage reaction, such as methyl ethyl ketone, cyclohexanone, phenol or cyclohexylbenzene, to assist in heat removal. The cleavage reaction can be conveniently conducted in a catalytic distillation unit.

The catalyst employed in the cleavage step can be a homogeneous catalyst or a heterogeneous catalyst.

Suitable homogeneous cleavage catalysts include sulfuric acid, perchloric acid, phosphoric acid, hydrochloric acid, and p-toluenesulfonic acid. Ferric chloride, boron trifluoride, sulfur dioxide, and sulfur trioxide are also effective homogeneous cleavage catalysts. The preferred homogeneous cleavage catalyst is sulfuric acid, with preferred concentrations in the range of 0.05 wt % to 0.5 wt %. For a homogeneous acid catalyst, a neutralization step preferably follows the cleavage step. Such a neutralization step typically involves contact with a basic component, with subsequent removal of a salt-enriched phase by decanting or distillation.

A suitable heterogeneous catalyst for use in the cleavage of cyclohexylbenzene hydroperoxide includes a clay, such as an acidic montmorillonite silica-alumina clay, as described in U.S. Pat. No. 4,870,217, or a faujasite molecular sieve as described in WO 2012/145031.

The effluent from the cleavage reaction comprises phenol and cyclohexanone in substantially equimolar amounts and, depending on demand, the cyclohexanone can be sold or can be dehydrogenated into additional phenol. Any suitable dehydrogenation catalyst can be used in this reaction, such as the dehydrogenation catalyst or a variation of the catalyst described herein. Suitable conditions for the dehydrogenation step comprise a temperature of 250° C. to 500° C. and a pressure of 0.01 atm to 20 atm (1 kPa to 2030 kPa), such as a temperature of 300° C. to 450° C. and a pressure of 1 atm to 3 atm (100 kPa to 300 kPa).

The invention will now be more particularly described with reference to the following non-limiting examples.

### EXAMPLES

In the examples, a catalyst precursor comprising Sn and Pt (and essentially free of a third meal) deposited on a SiO₂ inorganic support, was prepared by the following steps:

Step 1: an extruded silica support having a composition >93% SiO₂, a B.E.T. surface area >90 m²/g, and no measurable acidity as determined by Temperature Programmed Ammonia Adsorption was impregnated with 0.15 wt % tin using a an aqueous solution of stannous chloride to obtain a first impregnated support;

Step 2: the first impregnated support was dried at a temperature below 200° C. to reduce the water content to a level less than 5 wt % to obtain a first dried support;

Step 3: the first dried support was impregnated with 1 wt % platinum using a second aqueous solution of tetraamine platinum hydroxide to obtain a second impregnated support;

Step 4: the second impregnated support was dried at a temperature below 200° C. to reduce the water content to a level less than 5 wt % to obtain a catalyst precursor; and, optionally,

Step 5: the second dried support was calcined at a temperature of 350° C. for 1 hour to obtain a calcined catalyst precursor.

Using Wavelength Dispersive X-Ray Fluorescence spectroscopy (“XRF”), the chloride content of the catalyst precursor following steps (2), (4), and (5) was determined to be 0.16 wt %, 0.14 wt %, and 0.11 wt %, respectively. In comparison, the theoretical chloride content of the catalyst precursor, on the assumption that every C1 atom present during the preparation steps would be present in the prepared precursor, was 0.134 wt %. The XRF analysis was performed using a Bruker S8 Tiger Wavelength Dispersive X-ray Fluorescence (WDXRF) 3 kW System Spectrometer, wherein the sample was ground into a powder prior to analysis.

The catalyst precursor obtained from Step (4) or the calcined catalyst precursor obtained by Step (5) prepared in accordance with the above steps were subjected to various treatment options to remove unwanted ionic species, as described in the following examples and comparative examples.

### Comparative Example 1

The catalyst precursor obtained from Step (4) above was calcined in dry air at 400° C. for 1 hour. Elemental analysis data of the material obtained via XRF are shown in Table 1. The data in Table 1 suggest that calcining the catalyst precursor in air at a higher temperature than the calcination procedure of Step (5) had a limited impact on chloride and sulfur removal.

### Comparative Example 2

The catalyst precursor obtained from Step (4) above was calcined in hydrogen using a temperature procedure comprising ramping from 23° C. to 400° C. at 5° C./min, then holding at 400° C. for 3 hours. Elemental analysis data of the material obtained via XRF are shown in Table 1. The data in Table 1 suggest that that replacing the air atmosphere of the calcination procedure of Step (5) with a hydrogen atmosphere had a negative impact on both Sn and Pt retention, i.e., resulted in a large decrease in Sn and Pt contents on the support, and had a limited impact on chloride and sulfur removal.

### Example 1

The calcined catalyst precursor obtained from Step (5) above was calcined in hydrogen using the same temperature procedure as Comparative Example 2. Elemental analysis data of the material obtained via XRF are shown in Table 1. The data in Table 1 demonstrate that the combination of calcining the catalyst precursor in an air atmosphere in accordance with Step (5) followed by calcination in hydrogen under the conditions of this example was effective in removing around 27% of the C1 present in the catalyst precursor.

### Example 2

The calcined catalyst precursor obtained from Step (5) above was calcined in hydrogen using a temperature procedure comprising ramping from 23° C. to 500° C. at 1° C./min, then holding at 500° C. for 18 hours. Elemental analysis data of the material obtained via XRF are shown in Table 1. The data in Table 1 demonstrate that the combination of calcining the catalyst precursor in an air atmosphere in accordance with Step (5) followed by calcination in hydrogen under the conditions of this example was effective in removing around 33% of the C1 present in the catalyst precursor and around 85% of the sulfur.

### Example 3

The calcined catalyst precursor obtained from Step (5) above was calcined in hydrogen using a temperature procedure comprising ramping from 23° C. to 450° C. at 1° C./min, then holding at 450° C. for 36 hours. Elemental analysis data of the material obtained via XRF are shown in Table 1. The data in Table 1 demonstrate that the combination of calcining the catalyst precursor in an air atmosphere in accordance with Step (5) followed by calcination in hydrogen under the conditions of this example was effective in removing around 40% of the C1 present in the catalyst precursor and around 55% of the sulfur.

### Example 4

The calcined catalyst precursor obtained from Step (5) above was calcined in hydrogen using a temperature procedure comprising ramping from 23° C. to 350° C. at 2.8° C./min, then holding at 350° C. for 3 hours. Elemental analysis data of the material obtained via XRF are shown in Table 1. The data in Table 1 demonstrate that the combination of calcining the catalyst precursor in an air atmosphere in accordance with Step (5) followed by calcination in hydrogen under the conditions of this example resulted in removing around 35% of the C1 present in the catalyst precursor.

### Example 5

The calcined catalyst precursor obtained from Step (5) was washed with de-ionized water at 23° C. for 1 hour, after which the water was drained and the catalyst was washed a second time with a fresh sample of deionized water at 23° C. for 1 hour. After the second wash, the water was drained from the catalyst and the catalyst was then dried in an oven at 121° C. for at least two hours until dry. XRF analysis of the material is shown in Table 1. The data in Table 1 demonstrate that water washing the catalyst precursor at ambient conditions resulted in removing 53% of the C1 and 86% of the sulfur present in the catalyst precursor with negligible loss of Sn and Pt.

### Example 6

The calcined catalyst from Step (5) above was washed with deionized water at 60° C. for 1 hour after which the water was drained and the catalyst washed a second time with a fresh sample of deionized water at 60° C. for 1 hour. After the second wash, the water was drained from the catalyst and the catalyst was then dried in an oven at 121° C. for at least two hours until dry. XRF analysis of the material is shown in Table 1. The data in Table 1 demonstrate that water washing the catalyst precursor 60° C. resulted in removing 72% of the C1 and 87% of the sulfur present in the catalyst precursor with negligible loss of Sn and Pt.

TABLE 1
Dehydrogenation Catalyst Compositions

| | | | Cl | | Na |
|---|---|---|---|---|---|
| Example | Sn (wt %) | Pt (wt %) | (wt %) | S (wt %) | (wt %) |
| Calcined Catalyst | 0.13 | 0.95 | 0.11 | 0.19 | 0.35 |
| Precursor | | | | | |
| Comparative 1 | 0.15 | 0.98 | 0.11 | 0.13 | N.A. |
| Comparative 2 | 0.07 | 0.43 | 0.11 | 0.12 | 0.33 |
| 1 | 0.11 | 0.83 | 0.08 | 0.14 | 0.31 |
| 2 | 0.16 | 0.97 | 0.08 | 0.03 | 0.33 |
| 3 | 0.17 | 1.01 | 0.07 | 0.05 | 0.31 |
| 4 | 0.15 | 0.98 | 0.07 | 0.12 | 0.33 |
| 5 | 0.15 | 0.95 | 0.05 | 0.03 | 0.13 |
| 6 | 0.16 | 0.95 | 0.03 | 0.02 | 0.14 |

It can be seen from the foregoing examples that the dehydrogenation catalyst preparation methods of the present disclosure results in significantly reduced levels of impurities, such as C1, S, Na, while also improving retention of the desired dehydrogenation metals.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention. All documents described herein are incorporated by reference herein, including any priority documents and/or testing procedures to the extent they are not inconsistent with this text.

1. A method of preparing a dehydrogenation catalyst, the method comprising the steps of:
a) providing a catalyst precursor comprising (i) an inorganic support comprising silica (ii) a first metal selected from Group 14 of the Periodic Table of Elements and (iii) a second metal selected from Groups 6 to 10 of the Periodic Table of Elements;
b) calcining the catalyst precursor at a temperature of from 200° C. to 700° C. in an oxygen-containing atmosphere to obtain a calcined catalyst precursor,
wherein the method further comprises one of the following steps c) through 0;
c) calcining the calcined catalyst precursor at a temperature ranging from 150° C. to 600° C. in a hydrogen-containing atmosphere to obtain the dehydrogenation catalyst;
d) washing the calcined catalyst precursor with water at a temperature of below 100° C. to obtain the dehydrogenation catalyst;
e) calcining the calcined catalyst precursor at a temperature ranging from 150° C. to 600° C. in a hydrogen-containing atmosphere, followed by washing the calcined catalyst precursor with water at a temperature of below 100° C. to obtain the dehydrogenation catalyst; or
f) washing the calcined catalyst precursor with water at a temperature of below 100° C., followed by calcining the calcined catalyst precursor at a temperature ranging from 150° C. to 600° C. in a hydrogen-containing atmosphere to obtain the dehydrogenation catalyst.

2. The method of claim 1, wherein step d), e), or f) further comprises the step of drying the calcined catalyst precursor at a temperature ranging from 100° C. to 200° C. to obtain the dehydrogenation catalyst.

3. The method of claim 1, wherein the water of step d), e), or f) comprises deionized water.

4. The method of any one of claim 1, wherein step a) further comprises the following steps:
a1) impregnating a silica-containing support with a first solution containing a first metal selected from Group 14 of the Periodic Table of Elements to obtain a first impregnated support;
a2) impregnating the first impregnated support with a second solution containing a second metal selected from Group 6 to 10 of the Periodic Table of Elements to obtain a second impregnated support;
a3) drying the second impregnated support at a temperature of below 200° C. to obtain a dried second impregnated support; and
a4) calcining the dried second impregnated support at a temperature ranging from 200 to 700° C. to obtain the catalyst precursor.

5. The method of claim 1, wherein in step c), e), or f) the calcined catalyst precursor is calcined at a temperature ranging from 300 to 600° C.

6. The method of claim 5, wherein in step c), e), or f) the calcined catalyst precursor is calcined at a temperature ranging from 400 to 525° C.

7. The method of claim 1, wherein step d), e), or f) comprises washing the calcined catalyst precursor at least twice.

8. The method of claim 1, wherein in step d), e), or f) the washing is conducted at a temperature from 20 to 95° C.

9. The method of claim 1, wherein the first metal comprises tin.

10. The method of claim 1, wherein the source of the first metal is a chloride salt.

11. The method of claim 10, wherein the chloride salt is stannous chloride.

12. The method of claim 1, wherein the first metal is present in an amount ranging from 0.05 to 5 wt % based on the weight of the dehydrogenation catalyst.

13. The method of claim 1, wherein the second metal comprises platinum and/or palladium.

14. The method of claim 1, wherein the second metal is present in an amount ranging from 0.1 to 10 wt % based on the weight of the dehydrogenation catalyst.

15. The method of claim 1, wherein the silica-containing support comprises less than 0.5 wt % alumina based on the weight of the silica-containing support.

16. The method of claim 1, wherein the dehydrogenation catalyst has a halogen content of less than 0.1 wt % based on the weight of the dehydrogenation catalyst.

17. The method of claim 1, wherein the dehydrogenation catalyst comprises less than 0.20 wt % sulfur based on the weight of the dehydrogenation catalyst and/or less than 0.15 wt % sodium based on the weight of the dehydrogenation catalyst.

18. A dehydrogenation catalyst comprising (i) from 0.05 to 5 wt % of a first metal oxide based on the weight of the dehydrogenation catalyst, wherein the first metal oxide comprises a metal selected from Group 14 of the Periodic Table of Elements and (ii) from 0.1 to 10 wt % of a second metal oxide based on the weight of the dehydrogenation catalyst, wherein the second metal oxide comprises a metal selected from Groups 6-10 of the Periodic Table of Elements, wherein the first metal oxide and the second metal oxide are deposited on a silica-containing support, and wherein the dehydrogenation catalyst has a halogen content of less than 0.1 wt % based on the weight of the dehydrogenation catalyst.

19. The dehydrogenation catalyst of claim 18, wherein the first metal comprises tin.

20. The dehydrogenation catalyst of claim 18, wherein the second metal comprises platinum and/or palladium.

21. The dehydrogenation catalyst of claim 18, wherein the dehydrogenation catalyst comprises less than 0.10 wt % chlorine based on the weight of the dehydrogenation catalyst.

22. The dehydrogenation catalyst of claim 18, wherein the dehydrogenation catalyst comprises less than 0.20 wt % sulfur based on the weight of the dehydrogenation catalyst and/or less than 0.15 wt % sodium based on the weight of the dehydrogenation catalyst.

23. The dehydrogenation catalyst of claim 18, wherein the silica-containing support comprises less than 0.5 wt % alumina based on the weight of the silica-containing support.

24. A dehydrogenation process comprising the step of contacting a dehydrogenation feed containing cyclohexane and/or methylcyclopentane with the dehydrogenation catalyst prepared according to the method of any one of claims 1 to 17 or the dehydrogenation catalyst according to claim 18.

25. The dehydrogenation process of claim 24, wherein the dehydrogenation feed is obtained by:
a) contacting benzene and hydrogen with a hydroalkylation catalyst under a hydroalkylation conditions effective to convert benzene to cyclohexylbenzene, and cyclohexane and/or methylcyclopentane; and
b) separating at least a portion of the cyclohexane and/or methylcyclopentane from step a) to form the dehydrogenation feed.
